(19) United States
(12) Patent Application Publication
(10) Pub. No.: US 2004/0166179 A1
(43) Pub. Date: Aug. 26, 2004

(54) Dietary supplements from wine vinasses and relevant production process

(76) Inventors: Piergiorgio Anzaghi, Milano (IT); Rosanna Stefli, Pavia (IT)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/484,433

(22) PCT Filed: May 27, 2002
(86) PCT No.: PCT/EP02/05785

(51) Int. Cl. A61K 35/78

(52) U.S. Cl. 424/729; 424/766; 424/732; 424/764

(57) ABSTRACT

The present invention refers to a dietary supplement containing all of the natural components of wine, except for the volatile ones, in particular ethanol. Said dietary supplement is suitable for oral administration and contains antioxidant complexes present in wine vinasses combined with one or more bioavailability promoters. A preferred embodiment of the invention consists in a dietary supplement provided as solid or liquid formulation allowing for avoidance of wine consumption while maintaining all of the beneficial components, in particular the antioxidant ones.

### FIELD OF THE INVENTION

The present invention refers to antioxidant complexes derived from wine vinasses, wherefrom solid, semisolid or liquid formulations to be orally used as dietary supplements have been prepared. Said formulations comprise the same antioxidant complexes comprising polyphenolic compounds as contained in wine, e.g. resveratrols, and bioflavonoids, e.g. anthocyanins and polyphenols, but do not contain ethyl alcohol. Therefore, the said formulations do not present the hepatic and central toxicity problems caused by drinking wine to excess while providing for the well known benefits attributed to wine's natural constituents.

### PRIOR ART

Fruit, vegetables and beverages derived therefrom contain important constituents of the non-energetic diet displaying -antioxidant activity. More than 300 organic compounds belonging to the classes of carboxylic acids, mono- and disaccharides, amines, polyphenolic compounds, volatile compounds and pigments have been identified in wine. The major source of antioxidant activity are the polyphenolic compounds, which also affect the wine taste and colour. Particularly important are flavonoids, including catechins (catechin, epicatechin), flavone glycosides, flavonols (myricetin, quercetin, rutin, campherol, isoramnetin), flavanones, anthocyanins (delphinin, cyanin, petunin, peonin, malvin) and relevant anthocyanidins, and stilbenes (cis and trans resveratrols and glycosides thereof) present at higher concentrations in red grape skins and seeds, and in red wine.

Wine also contains carboxylic acids, such as for example citric and tartaric acid; benzoic acids, e.g. gallic acid, protocatechuic acid, vanillic and hydroxybenzoic acids; cinnamic acids, e.g. caffeic, cumaric, ferulic acids and others (M. Calull et al., J. Chromatogr., 590, 212-22, 1992; F. Mattivi, G. Nicolini, Biofactors, 6, 445-448,1997; E. N. Frankel et al., J. Agric. Food Chem., 43, 890-894, 1995).

A great number of benefits are brought about by the phenolic groups, due to their antioxidant, free-radical-inhibitory and metal sequestering activity (Catherine A. Rice-Evans et Lester Packer, Flavonoids in Health and Disease, Marcel Dekker, NY, 1998). Said groups protect man against cardiovascular diseases and thromboses caused by an excess of free oxygen radicals. Resveratrols, in particular, can inhibit platelet aggregation (R. J. Gryglewski et al., Biochem. Pharmacol., 36, 317-322, 1987) and prevent oxidation of low-density lipoproteins (LDL) (E. N. Frankel et al., Lancet, 341, 454-457, 1993). Furthermore, thanks to the presence of the aforementioned compounds, the moderate consumption of wine can increase the antioxidant capacity of human serum (Whitehead et al, Clin. Chem., 41, 32-35, 1995), can increase the plasmatic level of α-tocopherol and retinol (P. Simonetti et al., Alcohol Clin. Exp.Res., 19 (2), 517-522, 1995), and reduce fibrinogen levels (N. Pellegrini et al., Eur.J.Clin. Nutr., 50, 209-213, 1996). Finally, it has been found that a glass of red wine provides the organism with a much greater amount of flavonoids than that supplied by vegetables (P. G. Pietta et al. Dietary flavonoids and oxidative stress in “Natural antioxidants and food quality in atherosclerosis and cancer prevention”, J. T. Kumpfalien, Cambridge, 249-255, 1996), but that, especially in the case of heavy consumption of wine, alcohol causes considerable untoward side effects (M. Gronbaek et al., Biochem. Pharmacol., 36, 317-322,1987).

It is, therefore, clear that alcohol contributes in turn to the beneficial effect associated with wine consumption, as it secures the solubility of antioxidant complexes—in particular polyphenols—in the intestine environment (Goldberg, Clin. Chem. 41, 14-16, 1995) and that the bioavailability of the antioxidant complexes, in particular of the polyphenols present in grapes (or in the juice, skins and seeds thereof) is lower than that of the same polyphenols contained in wine.

It is therefore an object of the present invention to provide a food-grade substance capable of fully replacing a “daily” glass of wine—recommended in medical literature—whenever wine consumption is not advisable due to dietetic reasons or is forbidden by religious regulations.

It is a further object of the present invention to provide an alcohol-free, in particular ethyl alcohol-free, dietary supplement capable of supplying the organism with the antioxidant complexes commonly contained in wine, which are highly useful to the organism itself.

It is a still further object of the present invention to provide a dietary supplement bringing about an absorption of the antioxidant complexes commonly contained in wine, which is constant in time.

It is another object of the present invention to provide a process of manufacture of a dietary supplement, which process uses cheap and easily available raw materials and does not alter the antioxidant complexes contained therein.

### SUMMARY OF THE INVENTION

The above and further objects—which will be better described hereinafter—are achieved through a dietary supplement obtained from wine vinasse. In particular, according to an aspect of the present invention, a dietary supplement from wine vinasse suitable for oral administration is provided. According to a further feature of the present invention, a process for the obtainment of a dietary supplement in solid or liquid formulation from wine vinasse is provided.

### DETAILED DESCRIPTION OF THE INVENTION

Vinasse is the aqueous residue resulting from the distillation of wine, intended for the production of tasty alcohol for the liquor industry. Vinasse is a waste matter to be disposed of. It still contains all aforementioned classes of compounds (carboxylic acids, mono- and disaccharides, amines, polyphenolic compounds and pigments), whereas only ethyl alcohol and, partly, the flavouring volatile compounds have been eliminated.

By way of example, one litre of red wine can averagely contain 0.6 to 11 mg resveratrols (depending on the zone of origin) and gives approx. 0.7 l vinasse with a residue of 0.5 to 2.5% by wt., containing most of the antioxidant complexes present in wine. All of the above compounds are potentially of great biological interest; however, once they are separated from the alcoholic fraction, they have such a reduced bioavailability that they of little use for the organism. That is the reason why wine vinasses or concentrates thereof cannot be used as dietary supplements capable of simulating the dietetic properties of wine.

It is an object of the present invention to overcome the considerable wasting caused by the non-usability of vinasses through the exploitation of the antioxidants contained therein and the elimination of the relevant disposal problem. Therefore, according to the present invention, the vinasses have been added with particular substances capable of increasing the solubility and absorption in vivo of their components (said substances are called “bioavailability promoters”), such as to restore all of the dietetic properties of wine. We have indeed surprisingly found that there is a series of compounds, heterogeneous with one another from a chemical standpoint, which have the specific ability of restoring (“promoting”) the bioavailability of the useful compounds contained in vinasses and, therefore, allow use of vinasses as antioxidant dietary supplements. According to the present invention, the absorption of the antioxidant complexes present in wine vinasses may be restored with bioavailability promoters selected from the group consisting of polysaccharides (such as for example dextrans, maltodextrins, and inulin) and amino acids such as for example glycine, proline, leucine, and lysine.

According to a preferred embodiment of the present invention, the absorption (and, consequently, the haematic levels) of the antioxidant complexes present in wine vinasse is rendered more constant in time by means of sustained release formulations. Such a constant absorption profile could be hardly obtained through wine consumption itself, since wine should be drunk in small quantities and continually in the space of 24 hours. Consequently, the present invention allows not only to simulate the whole dietetic properties of wine, but also to render the said properties available in a more uniform manner in time: the organism can thus better face the continuous exposure to radicals.

The Applicant has also developed processes for the preparation of solid compositions, which do not alter the active ingredients. The liquid forms are directly obtained from vinasses, preferably after addition of bioavailability promoters, followed by filtration.

The starting products utilised in the present invention are preferably marc-red and moderately sweet vinasses of red wine, whose resveratrols and anthocyans concentration is higher than that of white or rosé wines.

In the case of drinkable preparations, vinasses are added with polysaccharides, e.g. dextrans, maltodextrins or inulin, or else amino acids, e.g. such as for example glycine, proline, leucine, and lysine, as bioavailability promoters to increase the in vivo assimilation of dietetically precious compounds, i.e. of antioxidant complexes. Out of dextrans, dextran 5 (m.w. 5000) is preferably used, and out of maltodextrins, those having 9-12 dextrose equivalents (DE) are preferred, in particular Maltrin® M500. Especially the vinasses of white and rosé wines are optionally added e.g. with vitamin C or green tea, blueberry, strawberry or red currant extracts, which enhance the antioxidant capacity. If necessary, to improve the pleasant taste, vinasses are added with substances preferably but not compulsorily present in wine, e.g. organic acids, sugars and amines, colouring and flavouring agents like e.g. limonene, diethylsuccinate, hexyl acetate, trans-hexenol and/or citronellol. The solutions are then filtered through a 0.45 μm porous filter and poured into “drinkable” vials or tiny bottles.

In the case of solid preparations for packets, capsules and tablets, the aforesaid solutions containing bioavailability promoters are dried preferably by freeze-drying or spray-drying. With a view to improving granulation and compression processes, the solid residue is then mixed with the same raw materials as usually employed in food industry as diluents, binding agents, anticaking agents and absorbents. Alternatively, vinasses drying may also be carried out before addition of bioavailability promoters and/or optional additives.

In relation to the starting liquid vinasse, the bioavailability promoters used in the present invention are dextrans, inulin or maltodextrins at concentrations of 0.4% to 30% (g/100 ml), and glycine, proline, leucine or lysine at concentrations of 0.12% to 2% (g/100 ml). The optional antioxidants used, especially for vinasses from white or rosé wines, are blueberry dry extract, 25% in anthocyanidins, at concentrations of 0.015% to 0.1% (g/100 ml), decaffeinated green tea dry extract, 50% in polyphenols at concentrations of 0.1% to 2% (g/100 ml), currant dry extract, 3.8% in flavonoids, at concentrations of 0.013% to 0.08% (g/100 ml), and vitamin C at concentrations of 0.2% to 2% (g/100 ml).

For the preparation of solid forms, the starting solution or the dry residue are added with excipients, diluents, binding agents, such as for example lactose (qs) (preferably from 0.4% to 0.7% (g/l 00 ml) in the case of the solution or from 12% to 30% in the case of the dry residue); starch, e.g. from potatoes (qs) (preferably from 0.4% to 0,7% (g/100 ml) in the case of the solution or from 6% to 25% in the case of the dry residue); microcrystalline cellulose (qs) (preferably from 0.7% to 1% (g/100 ml) in the case of the solution or from 1% to 38% in the case of the dry residue); mannitol (qs) and/or silica (qs). In particular, lactose and cellulose allow a direct compression of powders or the preparation of a granulated product by the wet or dry method. In a preferred embodiment of the invention, also 10% to 50% hydroxypropyl methylcellulose, having a viscosity of 4000 cps, is used for the sustained release tablets coating.

For the drinkable solution, the use of a preservative, such as benzyl alcohol (0.5-1%) or sodium benzoate (o.02-0.5%) and a further addition of a stabiliser, e.g. citric or tartaric acid, already present in wine, is also envisaged.

Analytical Control

The following compounds were identified within vinasses as such, as well as within the antioxidant complexes obtained by dry concentration thereof: resveratrol, quercetin and catechin, total phenols and anthocyanins.

Total polyphenols were identified by a method developed at our laboratories, based on UV-VIS spectrometry. Red wine vinasses and complexes obtained therefrom were diluted up to 200 times with methanol, whereas the white wine ones were diluted up to 40 times. A catechin-methanol solution at a concentration of 10 mg/ml was used as a reference. Each determination was repeated 5 times.

The analysis showed an absorption spectrum between 200 and 500 nm for all samples with D.O. value at 280 nm. The total polyphenols content was calculated as catechin concentration (mg/1).

Resveratrols were instead determined using a liquid chromatograph comprising an UV/VIS detector, and a 100 CN 250×4 mm column (Lichrosphere). The mobile phase was water:acetronitrile:methanol (90:5:5) at a flow rate of 1 ml per minute. The wavelength was set at 306 nm. (D. M. Goldberg et al., J. Chromatogr. A 708, 89-98, 1995). The samples to be analysed were dissolved in alcohol and diluted with a 0.2 M phosphoric acid:acetonitrile solution (4:1).

For the determination of total anthocyans, use was made of a method capable of determining the concentration of same from the test sample absorbance variation resulting from the decolouration brought about by the reaction with sulphur dioxide. To this end, the sample was first diluted in ethanol and HCl; then, a part thereof was added with water and a part with a sodium bisulphite solution. The difference in absorbance between the two solutions allows for the calculation of the anthocyanes mg/l.

Quercetin and catechin were determined simultaneously by a method developed at our laboratories using a liquid chromatograph comprising a variable wavelength UV/VIS detector and a 125×4 mm column (Lichrosorb Diolo). The mobile phase was hexane:ethanol (70:30) acidified with phosphoric acid, at a flow rate of 0.8 ml per minute. The wavelength was set at 280 nm. The substances were diluted in ethyl alcohol to obtain solutions at a concentration of 10 mcg/ml; and 20 mcl of the same was injected.

The peaks were clearly distinct, the retention time being approx. 6 min for quercitin and approx. 13 min for catechin.

Antioxidant Capacity

The antioxidant capacity of vinasses and complexes was determined by the Miller-Rice-Evans method (N. J. Miller, C. Rice-Evans, Redox Rep., 2 (3), 161-171, 1996).

The chromogenic substance ABTS [2,2′-azinobis(3-ethyl-benzothiazoline-6-sulphonate] in the presence of potassium persulphate was converted into a blue-green monocationic radicalic form, ABTS⁻⁺. The addition of an antioxidant analogous to vitamin E, denominated Trolox, caused—in proportion to the concentration of same—the decolouration of the solution, whose absorbance value was spectrographically read at 734 nm. The antioxidant capacity (TAC) of vinasses and of the new products was determined by comparing the absorbance value of the radicalic solution contacted with Trolox and with the test sample; it is expressed as mM Trolox eq./kg.

Table 1 shows, by way of example, the concentrations of some polyphenolic compounds in red wine vinasses (Recioto, 1998 vintage), in a Recioto freeze-dried vinasse, in a spray-dried rosé vinasse, 1998 vintage, in vinasses of Pinot grigio of the Veneto region, 1999 vintage, and the antioxidant capacity of same.

TABLE 1

| | Resveratrol | Catechin | Quercetin | Total phenols | Anthocyans | TAC mM |
|---|---|---|---|---|---|---|
| Sample | mcg/ml | mcg/ml | mcg/ml | mcg/ml | mcg/ml | Trolox |
| Recioto | 3.7 | 1.9 | 0.02 | 24. | 88.9 | 3.9 |
| vinasses | | | | | | |
| Freeze- | 3.5 | 1.9 | 0.02 | 26 | 246 | 6.3 |
| dried | | | | | | |
| Example 3 | | | | | | |
| Atomised | 1.8 | 1.7 | 0.03 | 21 | 153 | 4.0 |
| Example 5 | | | | | | |
| Pinot | 0.05 | 0.02 | n.d. | 16.2 | n.d. | 0.6 |
| grigio | | | | | | |
| vinasses | | | | | | |

Experimental Part

The following examples illustrate the claimed invention. These examples are illustrative only; in no event are they to be regarded as limiting the scope of the invention, which is defined by the claims reported hereinafter.

### EXAMPLE 1

### Drinkable Solution of Red Wine Vinasse with Dextran

Red wine vinasses (1 l) of a winy and moderately sweet taste were added with dextran 5 (20 g; m.w. 5000), fructose (0.6 g), blueberry dry extract (0.15 g), sodium benzoate (50 mg) and citric acid (0.2 g). The resultant solution was filtered through a 0.45 μm porous filter and bottled. A beverage of pleasant taste having an antioxidant capacity equal to 4.12 mM Trolox was obtained.

### EXAMPLE 2

### Freeze-Dried White Wine Vinasse with Maltodextrin

White wine vinasses (1 l) were added with maltodextrin (100 g), i.e. Maltrin® M500, blueberry extract (1 g) and green tea extract (1 g). The resultant solution was filtered through a 0.45 μm porous filter and freeze-dried according to a cycle comprising the following temperatures: −35° C. for pre-freezing, −10° C. during freeze-drying, 0° C., +10° C. and 28° C. for drying. 7.4·10⁻² mbar vacuum was maintained.

The light pink granular powder obtained (117 g) had an antioxidant capacity equal to 4.2 mM Trolox.

### EXAMPLE 3

### Freeze-Dried Red Wine Vinasse with Maltodextrin

Red wine vinasses (1 l) were added with maltodextrin (110 g), i.e. Maltrin® M500, and blueberry extract (0.7 g). The resultant solution was filtered and freeze-dried as described in Example 2. The residue obtained (124.5 g), in the form of a hygroscopic marc-coloured powder, had an antioxidant capacity equal to 6.3 mM Trolox.

### EXAMPLE 4

### Freeze-Dried Red Wine Vinasse with Inulin and Glycine

Red wine vinasses (1 l) were added with inulin (5 g), glycine (1.8 g), green tea extract (2 g), and lactose (5 g). The resultant solution was filtered and freeze-dried according to the cycle described in Example 2. The dry residue obtained (27.4 g), in the form of a pink-violet compact powder, had an antioxidant capacity equal to 8.9 mM Trolox.

### EXAMPLE 5

### Spray-Dried Rosé Wine Vinasse with Dextran

In pink-coloured vinasses (1 l) were dissolved dextran 5 (5 g; m.w. 5000), blueberry extract (1 g), lactose (6 g), and starch (5 g). The resultant solution was filtered through a 0.45 μm porous filter and spray-dried by means of a mini spray-dryer (Mini Buchi): jet pressure 800 mbar, inlet T° 130° C., outlet T° 50° C., suction 100%.

The light pink granular powder obtained (32 g) had an antioxidant capacity equal to 4.0 mM Trolox.

### EXAMPLE 6

### Spray-Dried Red Wine Vinasse with Dextran

In dark red vinasses (1 l) were dissolved dextran 5 (4 g) (m.w. 5000), microcrystalline cellulose (8 g) and vitamin C (3 g). The resultant solution was filtered and dried as described in Example 5. The garnet-red fine powder obtained (29 g) had an antioxidant capacity of 4.5 mM Trolox.

### EXAMPLE 7

### Granulated Product Preparation

The product described in Example 3 was mixed with microcrystalline cellulose (2 g) and wet with a 5% PVP-ethanol solution (20 ml) to give a granulation mixture. The wet mass was sieved through a No. 25 sieve, dried in an air circulated oven at 35° C. and graded by size through the same sieve.

### EXAMPLE 8

### Capsules Preparation

The granulated product described in Example 7 was added with silica precipitate (0.4 g). The resultant product could fill one hundred and twenty 1 g capsules.

### EXAMPLE 9

### Packets Preparation

The granulated product described in Example 7 was added with citric acid (3 g), sodium bicarbonate (3 g), fructose (2 g), flavouring agent (1 g), and silica (0.4 g) to give a product to be subdivided into sixty 2 g packets.

### EXAMPLE 10

### Tablets Preparation

The product described in Example 4 was wet with a 4% PVP solution (10 ml). The wet mass was sieved through a No. 25 sieve, dried in an air circulated oven at 35° C. and graded by size through the same sieve. It was added with microcrystalline cellulose (1 g), fructose (1.5 g), flavouring agent (0.25 g), magnesium stearate (0.35 g) and talc (0.35 g), by simple mixing.

The powder was compressed with a manual press (pressure applied: 1000 kg), using 10 mm dia. hollow punches, to give fifty-five 0.5 g tablets.

### EXAMPLE 11

### Chewable Tablets Preparation

The product described in Example 4 was added with microcrystalline cellulose (1 g), fructose (2 g), flavouring agent (0.4 g), magnesium stearate (0.3 g) and talc (0.3 g), by simple mixing.

The powder was compressed by a press using 13 mm dia. flat punches, with cracker, to give twenty-five 1 g tablets.

### EXAMPLE 12

### Effervescent Tablets Preparation

The residue of Example 6 was mixed with lactose (4.15 g), starch (2 g), fructose (2 g), flavouring agent (0.5 g), enocyanin powder (10 mg), citric acid (2.5 g) and sodium bicarbonate (2.5 g). The powder was compressed with a press using 20 mm dia. flat punches. The tablets weighing 2 g were immediately enclosed in blister packs.

### EXAMPLE 13

Sustained Release Tablets Preparation

The product described in Example 4 was wet with a 4% PVP solution (10 ml). The wet mass was sieved through a No. 25 sieve, dried in an air circulated oven at 35° C. and graded by size through the same sieve. It was added with microcrystalline cellulose (1 g), magnesium stearate (0.35 g) and talc (0.35 g), by simple mixing.

The granulated product was compressed with a single manual press, using a 10 mm dia. hollow punch, to give 0.5 g tablets.

Hydroxypropyl methylcellulose (6 g), magnesium stearate (250 mg) and colloidal silica (150 mg) were mixed in a turbulator for a period of 15 min. The punch previously used was replaced by a 12 mm dia. hollow punch; then the single nuclei were coated with the mixed powder. In particular, the matrix was filled with powder (53 mg), a nucleus, further powder (53 mg) and, finally, was compressed.

The dual compression technique afforded 60 sustained release tablets, each weighing 0.6 g (±5%).

Industrial Applicability

The present invention provides compositions derived from wine vinasses added with bioavailability promoters, which may be used as dietary supplements capable of simulating the dietetic properties of wine, but without the toxic effects of alcohol. Furthermore, the sustained release compositions from wine vinasses make the beneficial effect of wine constant in time; furthermore, their effect simulates that produced by a continuous wine consumption.

The liquid and solid dietary supplements described may be added with further antioxidants, whenever necessary, in particular when derived from white or rose wine vinasses, which—as shown by the analytical data reported above—are rather poor in resveratrol.

The vinasses solid derivatives were obtained by freeze-drying and spray-drying processes, which are rapid, little expensive and do not deteriorate the antioxidant complexes.

The tablets, capsules or granulated products (preferably formulated for sustained release) are an alternative to drinkable solutions and are particularly appreciated by those who constantly use said compositions to react against radicals unbalance caused by: environmental pollution, tobacco smoke, stress, prolonged muscular efforts, incorrect diet, alcoholic drinks, some drugs, infective agents, inflammatory and neoplastic diseases.

1. A dietary supplement, suitable for oral administration, comprising antioxidant complexes derived from wine vinasses, in combination with one or more bioavailability promoters selected from the group consisting of polysaccharides and amino acids.

2. The dietary supplement as claimed in claim 1 in the form of a sustained release composition.

3. The dietary supplement as claimed in claim 1, wherein the antioxidant complexes are red wine vinasse derivatives.

4. The dietary supplement as claimed in claim 1, wherein the antioxidant complexes are white or rose wine vinasse derivatives.

5. The dietary supplement as claimed in claim 1, wherein the antioxidant additives are selected out of vitamin C, blueberry, currant, strawberry and/or green tea extracts.

6. The dietary supplement as claimed in claim 1, wherein the food-grade excipients are selected out of flavouring agents, preservatives, colouring and/or sweetening agents.

7. The dietary supplement as claimed in claim 6, wherein the flavouring agents are selected from the group consisting of limonene, diethylsuccinate, hexyl acetate, trans-hexenol and/or citronellol.

8. The dietary supplement as claimed in claim 1, wherein the antioxidant complexes contain one or more polyphenolic compounds consisting of one or more flavonoids selected from the group consisting of catechins, flavone glycosides, flavonols, flavanones, anthocyanins, anthocyanidins, and stilbenes.

9. The dietary supplement as claimed in claim 8, wherein catechins are selected between catechin and/or epicatechin.

10. The dietary supplement as claimed in claim 8, wherein flavonols are selected from the group consisting of myricetin, quercetin, rutin, campherol, isoramnetin.

11. The dietary supplement as claimed in claim 8, wherein anthocyanins are selected from the group consisting of delphinin, cyanin, petunin, peonin, malvin.

12. The dietary supplement as claimed in claim 8, wherein stilbenes are selected out of cis and trans resveratrols and glycosides thereof.

13. The dietary supplement as claimed in claim 1, wherein polysaccharides are selected from the group consisting of dextrans, maltodextrins and inulin.

14. The dietary supplement as claimed in claim 1, wherein the amino acids are selected from the group consisting of glycine, proline, leucine, and lysine.

15. The dietary supplement as claimed in claim 1 in the solid, semisolid or liquid form.

16. The dietary supplement as claimed in claim 1 in the form of capsules, pills, tablets, granules, syrup or drinkable vials.

17. The dietary supplement as claimed in claim 16 in the form of sustained release tablets.

18. Use of a combination of antioxidant complexes derived from wine vinasses with bioavailability promoters selected from the group consisting of polysaccharides and amino acids, for the preparation of a dietary supplement capable of simulating the dietetic properties of wine.

19. The use as claimed in claim 18, added with excipients suitable for the preparation of a sustained release supplement, whose effect simulates that produced by continual wine consumption.

20. Process for the preparation of a dietary supplement capable of simulating the dietetic properties of wine, containing a mix of the following ingredients:
(a) wine vinasses,
(b) one or more bioavailability promoters selected from the group consisting of polysaccharides and amino acids, preferably as defined in claims 13 and 14, and optionally of
(c) additives, e.g. excipients as defined in claim 19 and/or food-grade excipients as defined in claim 6.

21. The process as claimed in claim 20, wherein wine vinasses are dried by freeze-drying or spray-drying before or after mixing with the components identified under (b) and c).
